(19) United States
(12) Patent Application Publication
(10) Pub. No.: US 2020/0093882 A1
(43) Pub. Date: Mar. 26, 2020

(54) Vaginal Lubricant

(71) Applicants: Jason Leon Childress, Douglasville, GA (US); Camille Patrice Hutton Childress, Douglasville, GA (US)

(72) Inventors: Jason Leon Childress, Douglasville, GA (US); Camille Patrice Hutton Childress, Douglasville, GA (US)

(21) Appl. No.: 16/676,918

(22) Filed: Nov. 7, 2019

Related U.S. Application Data
(60) Provisional application No. 62/735,547, filed on Sep. 24, 2018.

(51) Int. Cl. A61K 36/886; A61K 35/644; A61K 47/18; A61K 47/12; A61K 47/36; A61P 15/02

(52) U.S. Cl. CPC A61K 36/886; A61K 35/644; A61P 15/02; A61K 47/12; A61K 47/36; A61K 47/183

(57) ABSTRACT

A method to manufacture a vaginal lubricant comprising combining water, aloe vera, disodium EDTA, sodium benzoate, potassium sorbate, and a phosphate buffer thereby forming a first phase solution. The heated first phase solution is cooled to thereby form a cooled first phase solution. Raw honey and the cooled first phase solution are combined to thereby form a second phase solution. Polysorbate 20 and a honey flavoring are combined to thereby form a fragrance phase solution. The fragrance phase solution and the second phase solution are combined to thereby form a third phase solution. The first phase solution includes 99.17% water, 0.219% aloe vera, 0.0547% disodium EDTA, 0.219% sodium benzoate, 0.219% potassium sorbate, and 0.01095% phosphate buffer. The second phase solution includes 8.065% raw honey. The fragrance phase solution includes 71.42% polysorbate 20 and 28.571% honey flavoring. The third phase solution includes 0.5% polysorbate and 0.2% honey flavoring.

### CROSS-REFERENCE TO RELATED APPLICATIONS

This application claims priority to U.S. Provisional Application No. 62/735,547 filed Sep. 24, 2018, which is hereby incorporated herein by reference.

### FIELD OF THE INVENTION

The present invention generally relates to lubricants and specifically to vaginal lubricants.

### BACKGROUND OF THE INVENTION

Vaginal health is an important part of the total well-being of women. First, vaginal health is important because bacterial colonies reside within the vaginal cavity. Here, changes to the vaginal environment due to, for example, intercourse and/or liquid exchange, may modify the composition of the bacterial colonies, which be detrimental to vaginal health. Second, the vaginal health may also be affected by sickness, disease, aging, a hysterectomy, or similar factors. For example, vaginal health issues can result in a decrease in vaginal moisture, which can affect a woman's ability to engage in sexual intercourse.

Natural lubrication produced by glands at the neck of the womb (i.e. the cervix) keeps the vagina supple and moist. The moisture traverses slowly down through the vagina, keeping it clean and removing dead cells. Vaginal moisture is slightly acidic, which helps to keep the area healthy, preventing infections such as, for example, thrush. While vaginal lubricants are currently available, many of them are configured to provide lubrication as opposed to improving vaginal health and wellness.

### BRIEF DESCRIPTION OF THE DRAWINGS

Some of the embodiments will be described in detail, with reference to the following figures, wherein like designations denote like members, wherein:

FIG. 1 depicts a method for manufacturing a vaginal lubricant according to some embodiments.

FIG. 2 depicts a method for manufacturing a vaginal lubricant according to some embodiments.

FIG. 3 depicts a method for manufacturing a vaginal lubricant according to some embodiments.

### DETAIL DESCRIPTIONS OF THE INVENTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of Steps of various processes or methods that are described herein are illustrative and not restrictive. Accordingly, it should be understood that, although Steps of various processes or methods may be shown and described as being in a sequence or temporal order, the Steps of any such processes or methods are not limited to being carried out in any particular sequence or order, absent an indication otherwise. Indeed, the Steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

All illustrations of the drawings are for the purpose of describing selected versions of the present invention and are not intended to limit the scope of the present invention.

Vaginal health is an important part of the total well-being of women. First, vaginal health is important because bacterial colonies reside within the vaginal cavity. Here, changes to the vaginal environment due to, for example, intercourse and liquid exchange, may modify the composition of the bacterial colonies, which can be detrimental to vaginal health. Second, the vaginal health may also be affected by sickness, disease, hysterectomy, or similar factors.

For example, vaginal health issues can result in a decrease in vaginal moisture, which can affect a woman's ability to engage in sexual intercourse. Natural lubrication produced by glands at the neck of the womb (i.e. the cervix) keeps the vagina supple and moist. The moisture traverses slowly down through the vagina, keeping it clean and removing dead cells. Vaginal moisture is slightly acidic, which helps to keep the area healthy, preventing infections such as, for example, thrush. While vaginal lubricants are currently available, many of them are configured to provide lubrication as opposed to improving vaginal health and wellness.

Vaginal lubricants disclosed herein seek to address the above issues by improving one or more health characteristics of a vagina. The instant vaginal lubricants are formed using edible organic products having antimicrobial effects. The antimicrobial aspect of the instant vaginal lubricant can improve the overall vaginal health by preventing the growth of harmful bacteria during or after sexual intercourse and achieves a cleansing effect. Second, the present invention also contains ingredients to soften and moisten the vaginal cavity for users experiencing vaginal dryness. This feature can enhance pleasure and prevent damage to the vaginal wall during sexual intercourse and improve the overall health of the vagina during and after sexual intercourse.

The instant disclosure seeks to provide vaginal lubricants to, for example, improve the health characteristics of a vagina and thereby facilitate copulation according to certain embodiments. The following description discloses relevant compositions as well as manufacturing and administration methods. Future embodiments may use different chemicals or compounds with similar property or add/remove chemical or compounds from the composition. The present embodiment comprises various methods of manufacturing vaginal lubricants, but future embodiments may modify the concentration or the method of preparation.

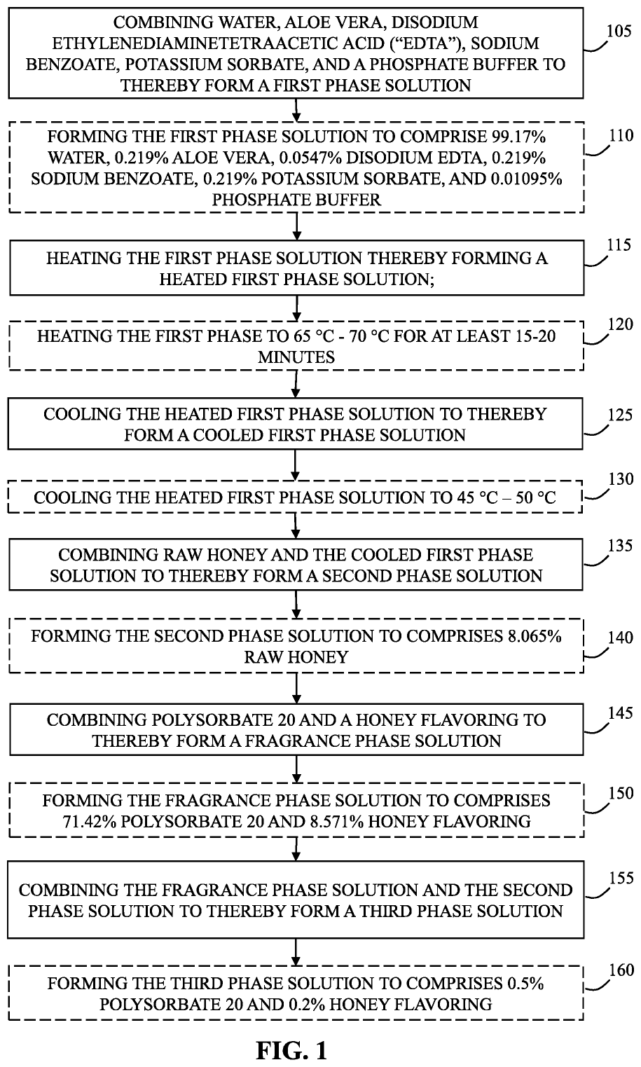

FIG. 1 depicts a method for manufacturing vaginal lubricants according to some embodiments. The method begins at Step 105 where water, aloe vera, disodium ethylenediaminetetraacetic acid (“EDTA”), sodium benzoate (a typical cosmetic preservative), potassium sorbate (a mild and natural preservative), and a phosphate buffer are combined to form a first phase solution. EDTA is a chemical that chelates (i.e. binds and holds) minerals and metals. In certain embodiments, the first phase solution is formed to include 99.17% water, 0.219% aloe vera, 0.0547% disodium EDTA, 0.219% sodium benzoate, 0.219% potassium sorbate, and 0.01095% phosphate buffer at Step 110. Aloe vera contains antioxidant, antimycoplasmic activity, as well as anthraquinones, which are compounds that actively heal and reduce pain through natural anti-inflammatory effects. In other embodiments, the aloe vera is an organic aloe vera powder. In yet still other embodiments, the phosphate buffer is a monophasic phosphate buffer.

Vaginal lubricants as well as the individual phase solutions disclosed herein are prepared in sterile environments (e.g., free of undesirable particles, chemicals, and/or bacteria). In certain embodiments, vaginal lubricants disclosed herein are manufactured according to Good Manufacturing Practices (“GMP”). To be sure, the US Food and Drug Administration (“FDA”) regulates vaginal lubricants as medical devices. GMP is a system that ensures products are consistently produced and controlled according to quality standards. GMP is designed to minimize the risks involved in any medical device production that cannot be eliminated through testing the final product.

At Step 115, the first phase solution is heated to thereby form a heated first phase solution. For example, at Step 120, heating the first phase preferably includes heating the first phase solution to 65° C.-70° C. for 15-20 minutes. At Step 125, the heated first phase solution is cooled to thereby form a cooled first phase solution. For example, at Step 130, the heated first phase solution is preferably cooled to 45° C.-50° C. At Step 135, raw honey and the cooled first phase solution are combined to thereby form a second phase solution. In other embodiments, at Step 140 the second phase solution is formed to include 8.065% raw honey. Raw honey has a variety of health benefits and medical uses. For example, raw honey can be pasteurized to improve color and texture, remove crystallization, and extend shelf life, but the beneficial nutrients are also destroyed in the process. In certain embodiments, the raw honey is organic raw honey (e.g., as defined by the United States Department of Agriculture).

At Step 145, polysorbate 20 and a honey flavoring are combined to thereby form a fragrance phase solution. For example, honey flavoring provides a psychologically beneficial aroma to vaginal lubricants (e.g., as opposed to medicinal aromas). In yet still other embodiments, the fragrance phase solution is formed to include 71.42% polysorbate 20 and 28.571% honey flavoring at Step 150. At Step 155, the fragrance phase solution and the second phase solution are combined to thereby form a third phase solution (i.e. the vaginal lubricant). In other embodiments, at Step 160, the third phase solution is formed to thereby include 0.5% polysorbate and 0.2% honey flavoring.

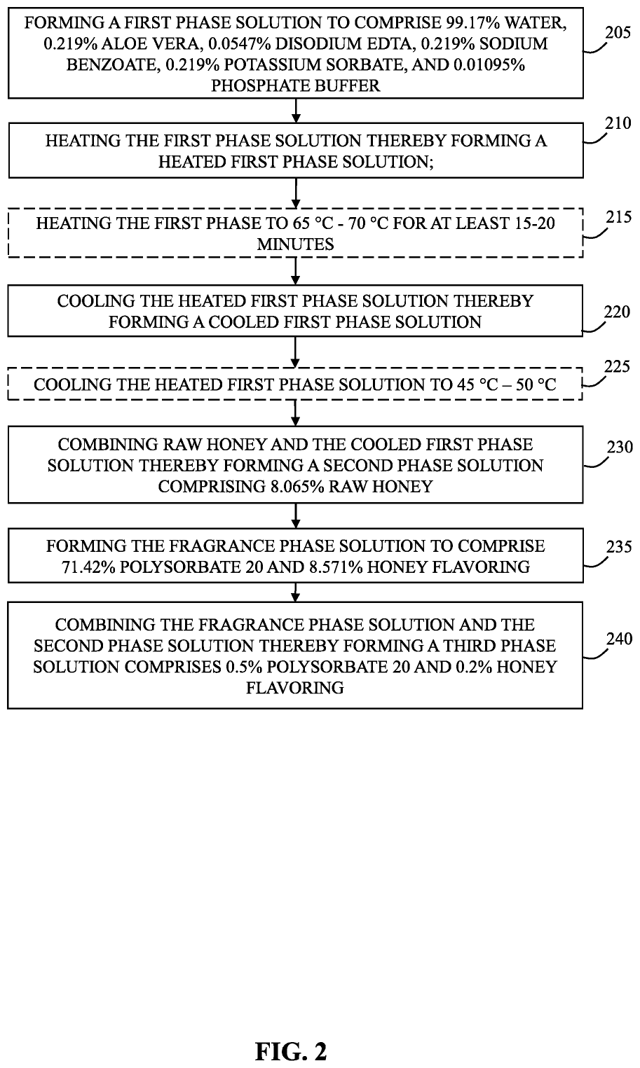

FIG. 2 depicts a method for manufacturing a vaginal lubricant according to some embodiments. The method begins at Step 205 where the first phase solution is formed to include 99.17% water, 0.219% aloe vera, 0.0547% disodium EDTA, 0.219% sodium benzoate, 0.219% potassium sorbate, and 0.01095% phosphate buffer. At Step 210, the first phase solution is heated to thereby form a heated first phase solution. In certain embodiments, at Step 215, the first phase solution is preferably heated to 65° C.-70° C. for 15-20 minutes. At Step 220, the heated first phase solution is cooled to thereby form a cooled first phase solution. In other embodiments, the heated first phase solution is preferably cooled to 45° C.-50° C. at Step 225.

At Step 230, raw honey and the cooled first phase solution are combined to thereby form a second phase solution that includes 8.065% raw honey. At Step 235, polysorbate 20 and a honey flavoring are combined to thereby form a fragrance phase solution that includes 71.42% polysorbate 20 and 28.571% honey flavoring. At Step 240, the fragrance phase solution and the second phase solution are combined to thereby form a third phase solution (i.e. the vaginal lubricant) that includes 0.5% polysorbate and 0.2% honey flavoring.

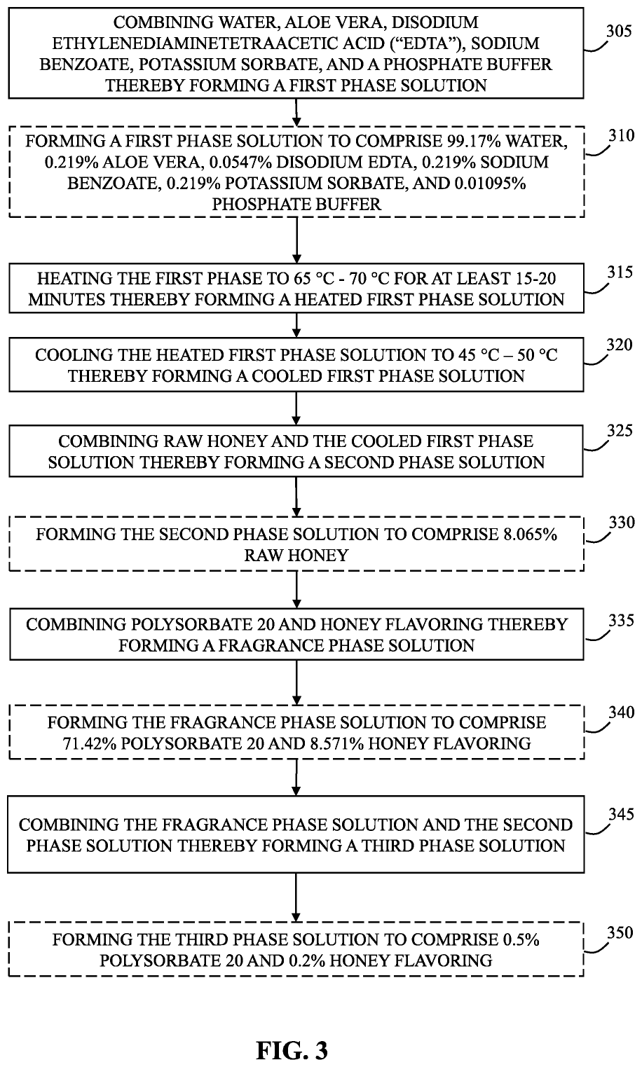

FIG. 3 depicts a method to manufacture a vaginal lubricant according to certain embodiments. The method begins at Step 305 where water, aloe vera, disodium ethylenediaminetetraacetic acid (“EDTA”), sodium benzoate, potassium sorbate, and a phosphate buffer are combined to form a first phase solution. In certain embodiments, the first phase solution is formed to include 99.17% water, 0.219% aloe vera, 0.0547% disodium EDTA, 0.219% sodium benzoate, 0.219% potassium sorbate, and 0.01095% phosphate buffer at Step 310. At Step 315, the first phase solution is heated to 65° C.-70° C. for 15-20 minutes. At Step 320, the heated first phase is cooled to 45° C.-50° C. At Step 325, raw honey and the cooled first phase solution are combined to thereby form a second phase solution. In some embodiments, at Step 330 the second phase solution is formed to include 8.065% raw honey.

At Step 335, polysorbate 20 and a honey flavoring are combined to thereby form a fragrance phase solution. In certain embodiments, the fragrance phase solution is formed to include 71.42% polysorbate 20 and 28.571% honey flavoring at Step 340. At Step 345, the fragrance phase solution and the second phase solution are combined to thereby form a third phase solution (i.e. the vaginal lubricant). In other embodiments, at Step 350, the third phase solution is formed to include 0.5% polysorbate and 0.2% honey flavoring.

In some embodiments, the vaginal lubricant (i.e. the third phase solution) is added to squeezable containers comprising applicators to facilitate its usage. For example, the applicator is inserted into the vaginal cavity (e.g., during a shower prior to copulation). Pressure is applied to the squeezable container to thereby introduce the vaginal lubricant to the vaginal cavity. Excess material can be removed from the cavity Not to be limited by theory, aloe vera softens the vaginal wall, the organic raw honey emits a psychologically pleasant scent, and the rest of the aforementioned components clean, moisturize and revitalize the vaginal wall, as well as improve the overall health of vagina.

Although the invention has been explained in relation to its preferred embodiment, it is to be understood that many other possible modifications and variations can be made without departing from the spirit and scope of the invention.

1. A method for manufacturing a vaginal lubricant comprising:
combining water, aloe vera, disodium ethylenediaminetetraacetic acid (“EDTA”), sodium benzoate, potassium sorbate, and a phosphate buffer thereby forming a first phase solution;
heating the first phase solution to thereby form a heated first phase solution;
cooling the heated first phase solution thereby forming a cooled first phase solution;
combining raw honey and the cooled first phase solution thereby forming a second phase solution;
combining polysorbate 20 and a honey flavoring thereby forming a fragrance phase solution; and
combining the fragrance phase solution and the second phase solution thereby forming a third phase solution.

2. The method of claim 1, wherein combining the water, the aloe vera, the EDTA, the sodium benzoate, the potassium sorbate, and the phosphate buffer to form the heated first phase solution comprises forming the heated first phase solution to comprise 99.17% water, 0.219% aloe vera, 0.0547% disodium EDTA, 0.219% sodium benzoate, 0.219% potassium sorbate, and 0.01095% phosphate buffer.

3. The method of claim 1, wherein combining the raw honey and the cooled first phase solution comprises forming the second phase solution to comprise 8.065% raw honey.

4. The method of claim 1, wherein combining the polysorbate 20 and the honey flavoring comprises forming the fragrance phase solution to comprise 71.42% polysorbate 20 and 28.571% honey flavoring.

5. The method of claim 1, wherein combining the fragrance phase solution and the second phase solution comprises forming the third phase solution to comprise 0.5% polysorbate 20 and 0.2% honey flavoring.

6. The method of claim 1, wherein heating the first phase solution comprises heating the first phase solution to 65° C.-70° C. for 15-20 minutes.

7. The method of claim 1, wherein cooling the heated first phase solution comprising cooling the heated first phase solution to 45° C.-50° C.

8. The method of claim 1, wherein the raw honey is an organic raw honey.

9. The method of claim 1, wherein the aloe vera is an organic aloe vera powder.

10. The method of claim 1, wherein the phosphate buffer is a monophasic phosphate buffer.

11. A method for manufacturing a vaginal lubricant comprising:
forming a first phase solution comprising the first phase solution comprises 99.17% water, 0.219% aloe vera, 0.0547% disodium EDTA, 0.219% sodium benzoate, 0. 219% potassium sorbate, and 0.01095% phosphate buffer;
heating the first phase solution thereby forming a heated first phase solution;
cooling the heated first phase solution thereby forming a cooled first phase solution;
combining raw honey and the cooled first phase solution thereby forming a second phase solution comprising 8.065% raw honey;
forming a fragrance phase solution comprising 71.42% polysorbate 20 and 28.571% honey flavoring; and
combining the fragrance phase solution and the second phase solution thereby forming a third phase solution comprising 0.5% polysorbate 20 and 0.2% honey flavoring.

12. The method of claim 11, wherein heating the first phase solution comprises heating the first phase solution to 65° C.-70° C. for 15-20 minutes.

13. The method of claim 12, wherein cooling the heated first phase solution comprising cooling the heated first phase solution to 45° C.-50° C.

14. The method of claim 13, wherein
the raw honey is an organic raw honey; and
the aloe vera is an organic aloe vera powder.

15. The method of claim 14, wherein the phosphate buffer is a monophasic phosphate buffer.

16. A method for manufacturing a vaginal lubricant comprising:
combining water, aloe vera, disodium EDTA, sodium benzoate, potassium sorbate, and a phosphate buffer to thereby form a first phase solution;
heating the first phase solution to 65° C.-70° C. for 15-20 minutes thereby forming a heated first phase solution;
cooling the heated first phase solution to 45° C.-50° C. thereby forming a cooled first phase solution;
combining raw honey and the cooled first phase solution thereby forming a second phase solution;
combining polysorbate 20 and a honey flavoring thereby forming a fragrance phase solution; and
combining the fragrance phase solution and the second phase solution thereby forming a third phase solution.

17. The method of claim 16, wherein combining the water, the aloe vera, the EDTA, the sodium benzoate, the potassium sorbate, and the phosphate buffer to form the heated first phase solution comprises forming the heated first phase solution to comprise 99.17% water, 0.219% aloe vera, 0.0547% disodium EDTA, 0.219% sodium benzoate, 0.219% potassium sorbate, and 0.01095% phosphate buffer.

18. The method of claim 17, wherein combining the raw honey and the cooled first phase solution comprises forming the second phase solution to comprise 8.065% raw honey.

19. The method of claim 18, wherein combining the polysorbate 20 and the honey flavoring comprises forming the fragrance phase solution to comprise 71.42% polysorbate 20 and 28.571% honey flavoring.

20. The method of claim 19, wherein combining the fragrance phase solution and the second phase solution comprises forming the third phase solution to comprise 0.5% polysorbate 20 and 0.2% honey flavoring.
